(19) United States
(12) Patent Application Publication
(10) Pub. No.: US 2018/0333144 A1
(43) Pub. Date: Nov. 22, 2018

(54) METHOD FOR SECURELY ATTACHING AND DETACHING OBTURATOR TO INTRODUCER

(71) Applicant: Devicor Medical Products, Inc., Cincinnati, OH (US)

(72) Inventors: Edward A. Rhad, Fairfield, OH (US); Bryan R. Keller, Loveland, OH (US)

(21) Appl. No.: 15/985,056

(22) Filed: May 21, 2018

Related U.S. Application Data
(60) Provisional application No. 62/509,485, filed on May 22, 2017.

(51) Int. Cl. A61B 10/02; A61B 5/055; A61B 17/34

(52) U.S. Cl. CPC A61B 10/02; A61B 2017/3411; A61B 17/3403; A61B 5/0555

(57) ABSTRACT

A targeting set for use with positioning a biopsy device relative to a patient includes an obturator, a sleeve, and a latching assembly. The obturator includes a seal cap. The sleeve includes a hub and defines a lumen configured to receive the obturator through the hub. The latching assembly includes an actuator arm attached to the seal cap and a catch positioned in the sleeve hub. The actuator arm includes a proximal end and a distal end adapted to mate with the catch. The proximal end of the actuator arm is configured to move radially inwardly to produce a radially outward movement of the distal end of the actuator arm to unlock the sleeve from the obturator.

### PRIORITY

This application claims priority to U.S. Provisional Patent App. No. 62/509,485 entitled “Method for Securely Attaching and Detaching Obturator to Introducer,” filed May 22, 2017, the disclosure of which is incorporated by reference herein.

### BACKGROUND

Biopsy samples have been obtained in a variety of ways in various medical procedures using a variety of devices. Biopsy devices may be used under stereotactic guidance, ultrasound guidance, MRI guidance, PEM guidance, BSGI guidance, or otherwise. For instance, some biopsy devices may be fully operable by a user using a single hand, and with a single insertion, to capture one or more biopsy samples from a patient. In addition, some biopsy devices may be tethered to a vacuum module and/or control module, such as for communication of fluids (e.g., pressurized air, saline, atmospheric air, vacuum, etc.), for communication of power, and/or for communication of commands and the like. Other biopsy devices may be fully or at least partially operable without being tethered or otherwise connected with another device.

Merely exemplary biopsy devices and biopsy system components are disclosed in U.S. Pat. No. 5,526,822, entitled “Method and Apparatus for Automated Biopsy and Collection of Soft Tissue,” issued Jun. 18, 1996; U.S. Pat. No. 6,017,316, entitled “Vacuum Control System and Method for Automated Biopsy Device,” issued Jan. 25, 2000; U.S. Pat. No. 6,086,544, entitled “Control Apparatus for an Automated Surgical Biopsy Device,” issued Jul. 11, 2000; U.S. Pat. No. 6,432,065, entitled “Method for Using a Surgical Biopsy System with Remote Control for Selecting an Operational Mode,” issued Aug. 13, 2002; U.S. Pat. No. 7,442,171, entitled “Remote Thumbwheel for a Surgical Biopsy Device,” issued Oct. 8, 2008; U.S. Pat. No. 7,938,786, entitled “Vacuum Timing Algorithm for Biopsy Device,” issued May 10, 2011; U.S. Pat. No. 8,083,687, entitled “Tissue Biopsy Device with Rotatably Linked Thumbwheel and Tissue Sample Holder,” issued Dec. 21, 2011; U.S. Pat. No. 8,206,316, entitled “Tetherless Biopsy Device with Reusable Portion,” issued Jun. 26, 2012; U.S. Pat. No. 8,241,226, entitled “Biopsy Device with Rotatable Tissue Sample Holder,” issued Aug. 14, 2012; U.S. Pat. No. 8,702,623, entitled “Biopsy Device with Discrete Tissue Chambers,” issued Apr. 22, 2014; U.S. Pat. No. 8,764,680, entitled “Handheld Biopsy Device with Needle Firing,” issued Jul. 1, 2014; U.S. Pat. No. 8,938,285, entitled “Access Chamber and Markers for Biopsy Device,” issued Jan. 20, 2015; U.S. Pat. No. 8,858,465, entitled “Biopsy Device with Motorized Needle Firing,” issued Oct. 14, 2014; and U.S. Pat. No. 9,326,755, entitled “Biopsy Device Tissue Sample Holder with Bulk Chamber and Pathology Chamber,” issued May 3, 2016. The disclosure of each of the above-cited U.S. Patents is incorporated by reference herein.

Additional exemplary biopsy devices and biopsy system components are disclosed in U.S. Pat. Pub. No. 2006/0074345, entitled “Biopsy Apparatus and Method,” published Apr. 6, 2006, now abandoned; U.S. Pat. Pub. No. 2009/0131821, entitled “Graphical User Interface For Biopsy System Control Module,” published May 21, 2009, now abandoned; U.S. Pat. Pub. No. 2010/0152610, entitled “Hand Actuated Tetherless Biopsy Device with Pistol Grip,” published Jun. 17, 2010, now abandoned; U.S. Pat. Pub. No. 2010/0160819, entitled “Biopsy Device with Central Thumbwheel,” published Jun. 24, 2010, now abandoned; and U.S. Pat. Pub. No. 2013/0324882, entitled “Control for Biopsy Device,” published Dec. 5, 2013, now abandoned. The disclosure of each of the above-cited U.S. Patent Application Publications, U.S. Non-Provisional Patent Applications, and U.S. Provisional Patent Applications is incorporated by reference herein.

In U.S. Pat. No. 7,831,290, entitled “MRI Biopsy Device Localization Fixture” issued Nov. 9, 2010, the disclosure of which is incorporated by reference herein, a localization mechanism, or fixture, is described that is used in conjunction with a breast coil for breast compression and for guiding a core biopsy instrument during prone biopsy procedures in both open and closed Magnetic Resonance Imaging (MRI) machines. The localization fixture includes a three-dimensional Cartesian positionable guide for supporting and orienting an MRI-compatible biopsy instrument, and, in particular, a cannula/sleeve to a biopsy site of suspicious tissues or lesions. Another merely illustrative localization mechanism used for guiding a core biopsy instrument is disclosed in U.S. Pat. No. 7,507,210, entitled “Biopsy Cannula Adjustable Depth Stop,” issued Mar. 24, 2009, the disclosure of which is incorporated by reference herein. The localization mechanism includes a grid plate configured to removably receive a guide cube capable of supporting and orienting an MRI-compatible biopsy instrument. For instance, a combination of an obturator and targeting cannula/sleeve may be introduced through a breast to a biopsy site via the guide cube, with proper positioning confirmed using MRI imaging. The obturator may then be removed and the needle of a biopsy device may then be inserted through the targeting cannula/sleeve to reach the targeted lesion.

While several systems and methods have been made and used for obtaining a biopsy sample, it is believed that no one prior to the inventor has made or used the invention described in the appended claims.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings, in which like reference numerals identify the same elements. In the drawings some components or portions of components are shown in phantom as depicted by broken lines.

FIG. 1 depicts a perspective view of a biopsy system including a control module remotely coupled to a biopsy device, and including a localization fixture with a lateral grid plate used in conjunction with a rotatable cube to position an obturator or a probe of the biopsy device to a desired insertion depth as set by a ring stop;

FIG. 2 depicts a perspective view of a breast coil receiving the localization fixture of FIG. 1;

FIG. 3 depicts a perspective view of the biopsy device inserted through the rotatable cube within the cube plate of the localization fixture attached to the breast coil of FIG. 2;

FIG. 4 depicts a perspective view of a two-axis rotatable guide cube of the biopsy system of FIG. 1;

FIG. 5 depicts a diagram of nine guide positions achievable by the two-axis rotatable guide cube of FIG. 4;

FIG. 6 depicts a perspective view of a two-axis rotatable guide cube into a lateral grid with the backing of the localization fixture of FIG. 1;

FIG. 7 depicts a perspective view of a obturator and cannula of the biopsy system of FIG. 1;

FIG. 8 depicts a perspective exploded view of the obturator and cannula of FIG. 7;

FIG. 9 depicts a perspective view of the obturator and cannula of FIG. 7 with a depth stop device of FIG. 1 inserted through the guide cube and grid plate of FIG. 6;

FIG. 10 depicts a perspective view of an exemplary targeting set that may be readily incorporated into the biopsy system of FIG. 1;

FIG. 11 depicts a perspective exploded view of the targeting set of FIG. 10;

FIG. 12 depicts a partial perspective view of a cannula of the targeting set of FIG. 10, with the cannula including a cylindrical hub having a pair of notches;

FIG. 13 depicts a rear elevational view of the cannula FIG. 12;

FIG. 14 depicts a side cross-sectional view of the cylindrical hub of the cannula of FIG. 12, with the cross-section taken along line 14-14 of FIG. 12;

FIG. 15 depicts a partial perspective view of an obturator of the targeting set of FIG. 10, with the obturator including a seal cap having a pair of latches;

FIG. 16 depicts a partial perspective view of the obturator of FIG. 15, with the obturator including a pair of user input features integral with the pair of latches;

FIG. 17 depicts a side cross-sectional view of the seal cap of FIG. 15, with the cross-section taken along line 17-17 of FIG. 15;

FIG. 18A depicts a side cross-sectional view of the targeting set of FIG. 10, with the obturator slidably advanced into the cannula and the pair of latches not coupled with the pair of notches, with the cross-section taken along line 18-18 of FIG. 10;

FIG. 18B depicts another side cross-sectional view of the targeting set of FIG. 10, with the obturator slidably advanced into the cannula and the pair of latches coupled with the pair of notches, with the cross-section taken along line 18-18 of FIG. 10; and

FIG. 18C depicts still another side cross-sectional view of the targeting set of FIG. 10, with the obturator slidably advanced into the cannula and the pair of latches decoupled from the pair of notches, with the cross-section taken along line 18-18 of FIG. 10.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

I. Overview of Exemplary MRI Biopsy Control Module

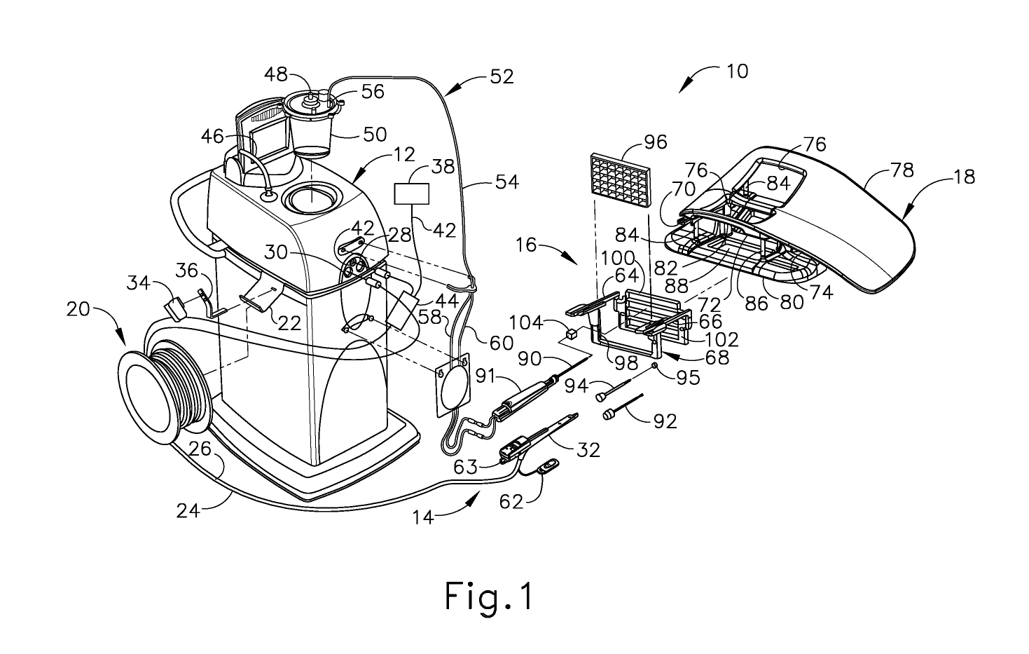

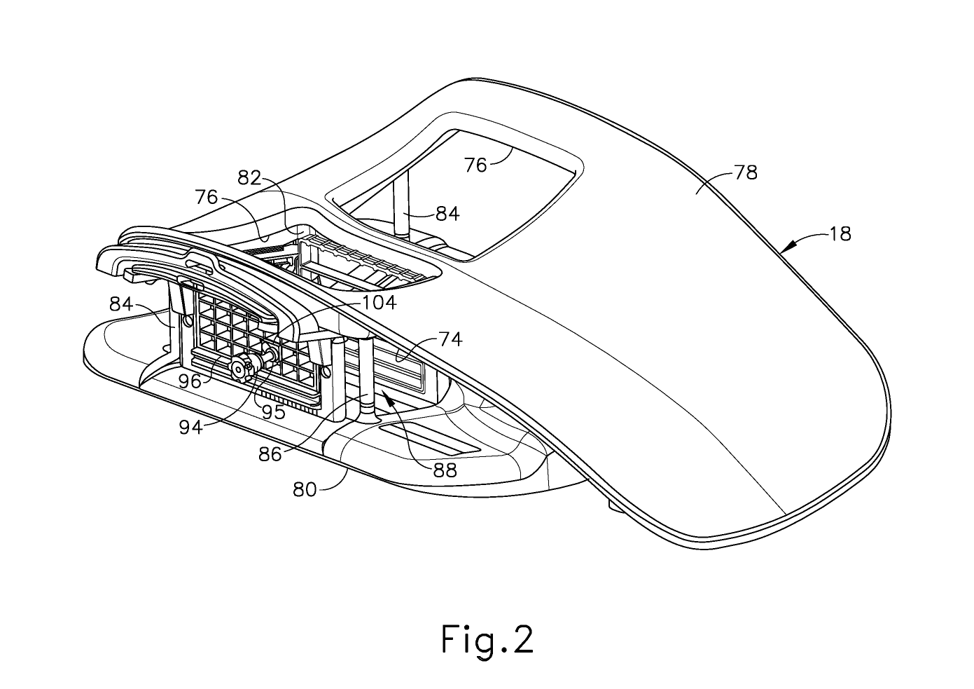

In FIGS. 1-3, an MRI compatible biopsy system (10) has control module (12) that may be placed outside of a shielded room containing an MRI machine (not shown) or at least spaced away to mitigate detrimental interaction with its strong magnetic field and/or sensitive radio frequency (RF) signal detection antennas. As described in U.S. Pat. No. 6,752,768, which is hereby incorporated by reference in its entirety, a range of preprogrammed functionality may be incorporated into control module (12) to assist in taking tissue samples. Control module (12) controls and powers biopsy device (14) that is used with localization assembly (15). Biopsy device (14) is positioned and guided by localization fixture (16) attached to breast coil (18) that may be placed upon a gantry (not shown) of a MRI or other imaging machine.

In the present example, control module (12) is mechanically, electrically, and pneumatically coupled to biopsy device (14) so that components may be segregated that need to be spaced away from the strong magnetic field and the sensitive RF receiving components of a MRI machine. Cable management spool (20) is placed upon cable management attachment saddle (22) that projects from a side of control module (12). Wound upon cable management spool (20) is paired electrical cable (24) and mechanical cable (26) for communicating control signals and cutter rotation/advancement motions respectively. In particular, electrical and mechanical cables (24, 26) each have one end connected to respective electrical and mechanical ports (28, 30) in control module (12) and another end connected to holster portion (32) of biopsy device (14). Docking cup (34), which may hold holster portion (32) when not in use, is hooked to control module (12) by docking station mounting bracket (36). It should be understood that such components described above as being associated with control module (12) are merely optional.

Interface lock box (38) mounted to a wall provides tether (40) to lockout port (42) on control module (12). Tether (40) is uniquely terminated and of short length to preclude inadvertent positioning of control module (12) too close to a MRI machine or other machine. In-line enclosure (44) may register tether (40), electrical cable (24) and mechanical cable (26) to their respective ports (42, 28, 30) on control module (12).

Vacuum assist is provided by first vacuum line (46) that connects between control module (12) and outlet port (48) of vacuum canister (50) that catches liquid and solid debris. Tubing kit (52) completes the pneumatic communication between control module (12) and biopsy device (14). In particular, second vacuum line (54) is connected to inlet port (56) of vacuum canister (50). Second vacuum line (54) divides into two vacuum lines (58, 60) that are attached to biopsy device (14). With biopsy device (14) installed in holster portion (32), control module (12) performs a functional check. Saline may be manually injected into biopsy device (14) or otherwise introduced to biopsy device (14), such as to serve as a lubricant and to assist in achieving a vacuum seal and/or for other purposes. Control module (12) actuates a cutter mechanism (not shown) in biopsy device (14), monitoring full travel of a cutter in biopsy device (14) in the present example. Binding in mechanical cable (26) or within biopsy device (14) may optionally monitored with reference to motor force exerted to turn mechanical cable (26) and/or an amount of twist in mechanical cable (26) sensed in comparing rotary speed or position at each end of mechanical cable (26).

Remote keypad (62), which is detachable from holster portion (32), communicates via electrical cable (24) to control panel (12) to enhance clinician control of biopsy device (14) in the present example, especially when controls that would otherwise be on biopsy device (14) itself are not readily accessible after insertion into localization fixture (16) and/or placement of control module (12) is inconveniently remote (e.g., 30 feet away). However, as with other components described herein, remote keypad (62) is merely optional, and may be modified, substituted, supplemented, or omitted as desired. In the present example, aft end thumbwheel (63) on holster portion (32) is also readily accessible after insertion to rotate the side from which a tissue sample is to be taken.

Of course, the above-described control module (12) is merely one example. Any other suitable type of control module (12) and associated components may be used. By way of example only, control module (12) may instead be configured and operable in accordance with the teachings of U.S. Pub. No. 2008/0228103, entitled “Vacuum Timing Algorithm for Biopsy Device,” published Sep. 18, 2008, the disclosure of which is incorporated by reference herein. As another merely illustrative example, control module (12) may instead be configured and operable in accordance with the teachings of U.S. Pat. No. 8,328,732, entitled “Control Module Interface for MRI Biopsy Device,” issued Dec. 11, 2012, the disclosure of which is incorporated by reference herein. Alternatively, control module (12) may have any other suitable components, features, configurations, functionalities, operability, etc. Other suitable variations of control module (12) and associated components will be apparent to those of ordinary skill in the art in view of the teachings herein.

II. Exemplary Localization Assembly

Left and right parallel upper guides (64, 66) of localization framework (68) are laterally adjustably received respectively within left and right parallel upper tracks (70, 72) attached to under side (74) and to each side of a selected breast aperture (76) formed in patient support platform (78) of breast coil (18). Base (80) of breast coil (18) is connected by centerline pillars (82) that are attached to patient support platform (78) between breast apertures (76). Also, a pair of outer vertical support pillars (84, 86) on each side spaced about a respective breast aperture (76) respectively define lateral recess (88) within which localization fixture (16) resides.

It should be appreciated that the patient's breasts hang pendulously respectively into breast apertures (76) within lateral recesses (88) in the present example. For convenience, herein a convention is used for locating a suspicious lesion by Cartesian coordinates within breast tissue referenced to localization fixture (16) and to thereafter selectively position an instrument, such as needle (90) of probe (91) that is engaged to holster portion (32) to form biopsy device (14). Of course, any other type of coordinate system or targeting techniques may be used. To enhance hands-off use of biopsy system (10), especially for repeated re-imaging within the narrow confines of a closed bore MRI machine, biopsy system (10) may also guide obturator (92) encompassed by cannula (94). Depth of insertion is controlled by depth stop device (95) longitudinally positioned on either needle (90) or cannula (94). Alternatively, depth of insertion may be controlled in any other suitable fashion.

This guidance is specifically provided by a lateral fence in the present example, depicted as grid plate (96), which is received within laterally adjustable outer three-sided plate bracket (98) attached below left and right parallel upper guides (64, 66). Similarly, a medial fence with respect to a medial plane of the chest of the patient, depicted as medial plate (100), is received within inner three-sided plate bracket (102) attached below left and right parallel upper guides (64, 66) close to centerline pillars (82) when installed in breast coil (18). To further refine the insertion point of the instrument (e.g., needle (90) of probe (91), obturator/cannula (92, 94), etc.), guide cube (104) may be inserted into grid plate (96).

In the present example, the selected breast is compressed along an inner (medial) side by medial plate (100) and on an outer (lateral) side of the breast by grid plate (96), the latter defining an X-Y plane. The X-axis is vertical (sagittal) with respect to a standing patient and corresponds to a left-to-right axis as viewed by a clinician facing the externally exposed portion of localization fixture (16). Perpendicular to this X-Y plane extending toward the medial side of the breast is the Z-axis, which typically corresponds to the orientation and depth of insertion of needle (90) or obturator/cannula (92, 94) of biopsy device (14). For clarity, the term Z-axis may be used interchangeably with “axis of penetration”, although the latter may or may not be orthogonal to the spatial coordinates used to locate an insertion point on the patient. Versions of localization fixture (16) described herein allow a non-orthogonal axis of penetration to the X-Y axis to a lesion at a convenient or clinically beneficial angle.

It should be understood that the above-described localization assembly (15) is merely one example. Any other suitable type of localization assembly (15) may be used, including but not limited to localization assemblies (15) that use a breast coil (18) and/or localization fixture (16) different from those described above. Other suitable components, features, configurations, functionalities, operability, etc. for a localization assembly (15) will be apparent to those of ordinary skill in the art in view of the teachings herein.

III. Exemplary Biopsy Device

As shown in FIG. 1, one version of biopsy device (14) may comprise holster portion (32) and probe (91). Exemplary holster portion (32) was discussed previously in the above section addressing control module (12). The following paragraphs will discuss probe (91) and associated components and devices in further detail.

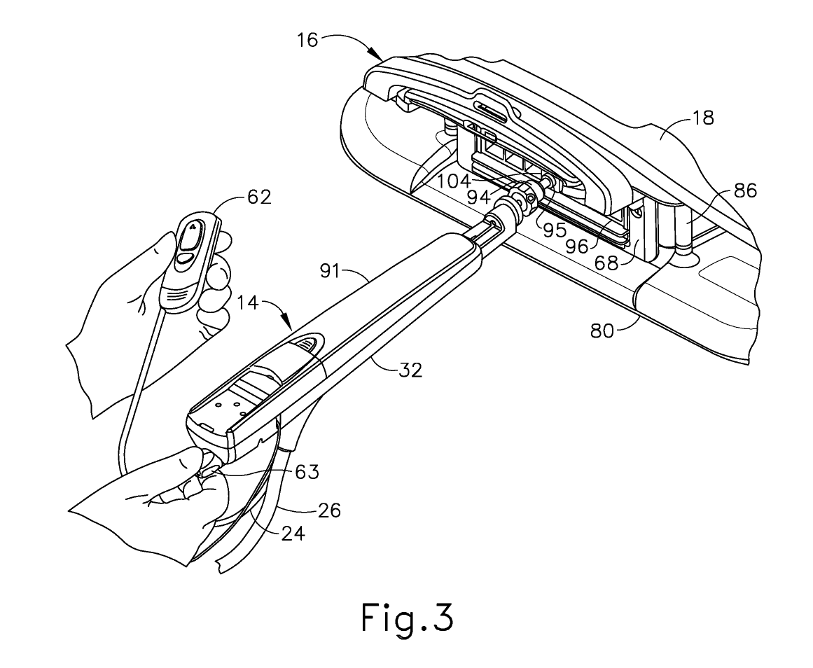

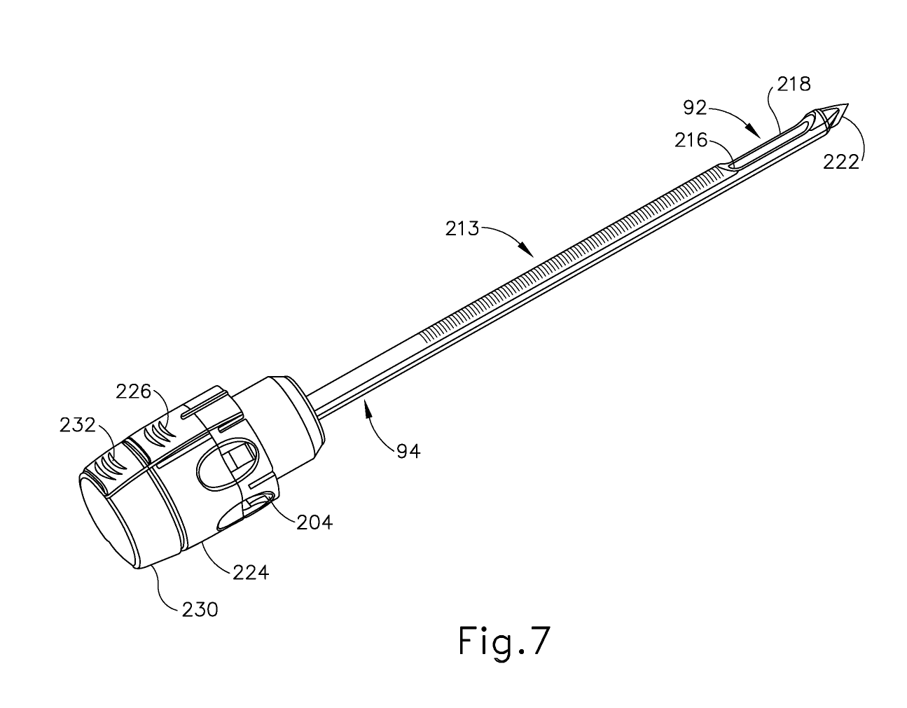

In the present example, a targeting set (89) comprising cannula (94) and obturator (92) is associated with probe (91). In particular, and as shown in FIGS. 7, 8, and 9, obturator (92) is slid into cannula (94) and the combination is guided through guide cube (104) to the biopsy site within the breast tissue. As shown in FIG. 3, obturator (92) is then withdrawn from cannula (94), then needle (90) of probe (91) is inserted in cannula (94), and then biopsy device (14) is operated to acquire one or more tissue samples from the breast via needle (90).

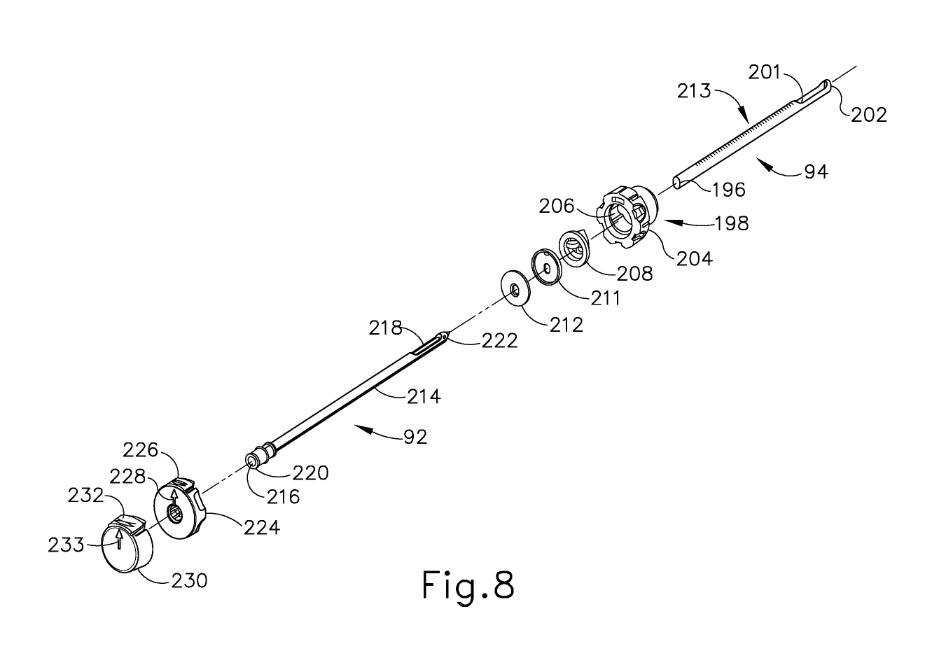

As best seen in FIG. 8, cannula (94) of the present example is proximally attached to cylindrical hub (198) and cannula (94) includes lumen (196) and lateral aperture (201) proximate to open distal end (202). Cylindrical hub (198) has exteriorly presented thumbwheel (204) for rotating lateral aperture (201). Cylindrical hub (198) has interior recess (206) that encompasses duckbill seal (208), wiper seal (211) and seal retainer (212) to provide a fluid seal when lumen (196) is empty and for sealing to inserted obturator (92). Longitudinally spaced measurement indicia (213) along an outer surface of cannula (94) visually, and perhaps physically, provide a means to locate depth stop device (95) of FIG. 1.

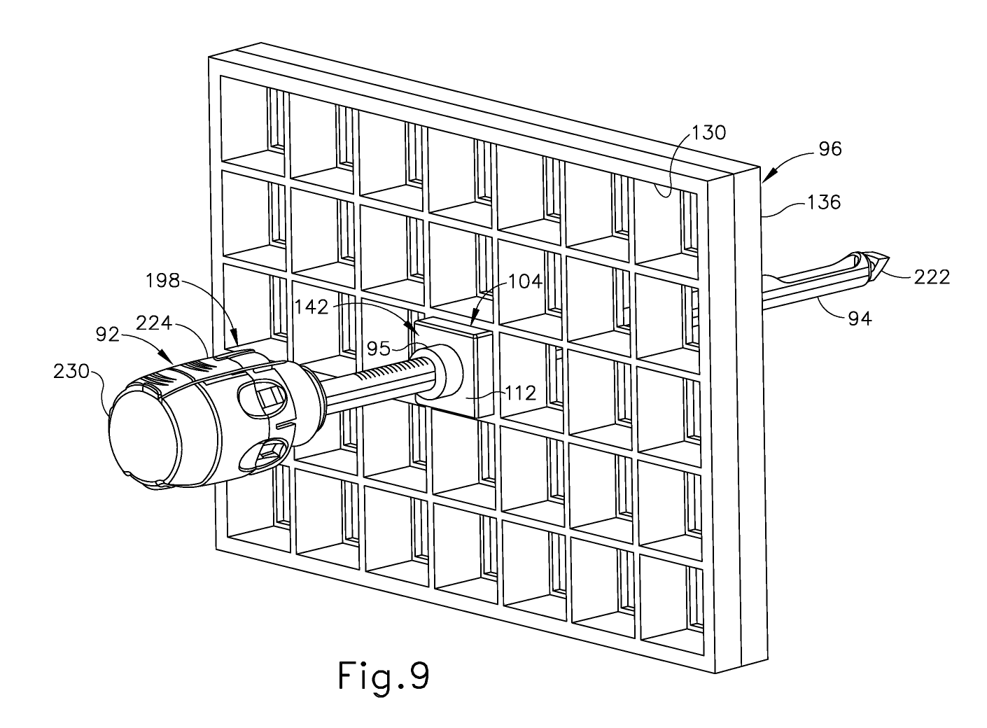

Obturator (92) of the present example incorporates a number of components with corresponding features. For instance, obturator (92) includes a shaft (214) that includes fluid lumen (216) that communicates between imageable side notch (218) and proximal port (220). Shaft (214) is longitudinally sized such that piercing tip (222) extends out of distal end (202) of cannula (94). Obturator thumbwheel cap (224) encompasses proximal port (220) and includes locking feature (226), which includes visible angle indicator (228), that engages cannula thumbwheel (204) to ensure that imageable side notch (218) is registered to lateral aperture (201) in cannula (94). Obturator seal cap (230) may be engaged proximally into obturator thumbwheel cap (224) to close fluid lumen (216). Obturator seal cap (230) of the present example includes locking or locating feature (232) that includes visible angle indicator (233) that corresponds with visible angle indicator (228) on obturator thumbwheel cap (224), which may be fashioned from either a rigid, soft, or elastomeric material. In FIG. 9, guide cube (104) has guided obturator (92) and cannula (94) through grid plate (96).

While obturator (92) of the present example is hollow, it should be understood that obturator (92) may alternatively have a substantially solid interior, such that obturator (92) does not define an interior lumen. In addition, obturator (92) may lack side notch (218) in some versions. Other suitable components, features, configurations, functionalities, operability, etc. for an obturator (92) will be apparent to those of ordinary skill in the art in view of the teachings herein Likewise, cannula (94) may be varied in a number of ways. For instance, in some other versions, cannula (94) has a closed distal end (202). As another merely illustrative example, cannula (94) may have a closed piercing tip (222) instead of obturator (92) having piercing tip (222). In some such versions, obturator (92) may simply have a blunt distal end; or the distal end of obturator (92) may have any other suitable structures, features, or configurations. Other suitable components, features, configurations, functionalities, operability, etc. for a cannula (94) will be apparent to those of ordinary skill in the art in view of the teachings herein. Furthermore, in some versions, one or both of obturator (92) or cannula (94) may be omitted altogether. For instance, needle (90) of probe (91) may be directly inserted into a guide cube (104), without being inserted into guide cube (104) via cannula (94).

Another component that may be used with probe (91) (or needle (90)) is depth stop device (95). Depth stop device (95) may be of any suitable configuration that is operable to prevent cannula (94) and obturator (92) (or needle (90)) from being inserted further than desired. For instance, depth stop device (95) may be positioned on the exterior of cannula (94) (or needle (90)), and may be configured to restrict the extent to which cannula (94) is inserted into a guide cube. It should be understood that such restriction by depth stop device (95) may further provide a limit on the depth to which the combination of cannula (94) and obturator (92) (or needle (90)) may be inserted into the patient's breast. Furthermore, it should be understood that such restriction may establish the depth within the patient's breast at which biopsy device (14) acquires one or more tissue samples after obturator (92) has been withdrawn from cannula (94) and needle (90) has been inserted in cannula (94). Exemplary depth stop devices (95) that may be used with biopsy system (10) are described in U.S. Pub. No. 2007/0255168, entitled “Grid and Rotatable Cube Guide Localization Fixture for Biopsy Device,” published Nov. 1, 2007, and incorporated by reference herein as mentioned previously.

In the present example, and as noted above, biopsy device (14) includes a needle (90) that may be inserted into cannula (94) after the combination of cannula (94) and obturator (92) has been inserted to a desired location within a patient's breast and after obturator (92) has been removed from cannula (94). Needle (90) of the present example comprises a lateral aperture (not shown) that is configured to substantially align with lateral aperture (201) of cannula (94) when needle (90) is inserted into lumen (196) of cannula (94). Probe (91) of the present example further comprises a rotating and translating cutter (not shown), which is driven by components in holster (32), and which is operable to sever tissue protruding through lateral aperture (201) of cannula (94) and the lateral aperture of needle (90). Severed tissue samples may be retrieved from biopsy device (14) in any suitable fashion.

It should be understood that although biopsy system (10) is discussed above as utilizing disposable probe assembly (91), other suitable probe assemblies and biopsy device assemblies may be utilized. By way of example only, other suitable biopsy devices may be configured and operable in accordance with at least some of the teachings of U.S. Pat. No. 8,206,316, entitled “Tetherless Biopsy Device with Reusable Portion,” issued Jun. 26, 2012, the disclosure of which is incorporated by reference herein; U.S. Pat. No. 8,277,394, entitled “Multi-Button Biopsy Device,” issued Oct. 2, 2012, the disclosure of which is incorporated by reference herein; and/or U.S. Pub. No. 2012/0065542, entitled “Biopsy Device Tissue Sample Holder with Removable Tray,” published Mar. 15, 2012, the disclosure of which is incorporated by reference herein. As yet another merely illustrative example, other suitable biopsy devices may be configured and operable in accordance with at least some of the teachings of U.S. Pub. No. 2010/0160824, the disclosure of which is incorporated by reference herein; U.S. Patent Pub. No. 2013/0144188, entitled “Biopsy Device with Slide-In Probe,” published Jun. 6, 2013, the disclosure of which is incorporated by reference herein; U.S. Patent Pub. No. 2013/0324882, entitled “Control for Biopsy Device,” published Dec. 5, 2013, the disclosure of which is incorporated by reference herein; U.S. Patent Pub. No. 2014/0039343, entitled “Biopsy System,” published Feb. 6, 2014, the disclosure of which is incorporated by reference herein; and/or U.S. Patent Pub. No. 2015/0065913, entitled “Tissue Collection Assembly for Biopsy Device,” published Mar. 5, 2015, the disclosure of which is incorporated by reference herein.

Still other suitable forms of biopsy devices that may be used in conjunction with the various alternative components of system (10) as described herein will be apparent to those of ordinary skill in the art.

IV. Exemplary Guide Cube

In some versions, a guide cube may comprise a body defined by one or more edges and faces. The body may include one or more guide holes or other types of passages that extend between faces of the guide cube and that may be used to guide an instrument such as a biopsy device (14) or a portion of a biopsy device (14) (e.g., needle (90) of biopsy device (14), a combination of cannula (94) and obturator (92), etc.). Guide cubes may be rotatable about one, two, or three axes to position the one or more guide holes or passages of the guide cube into a desired position.

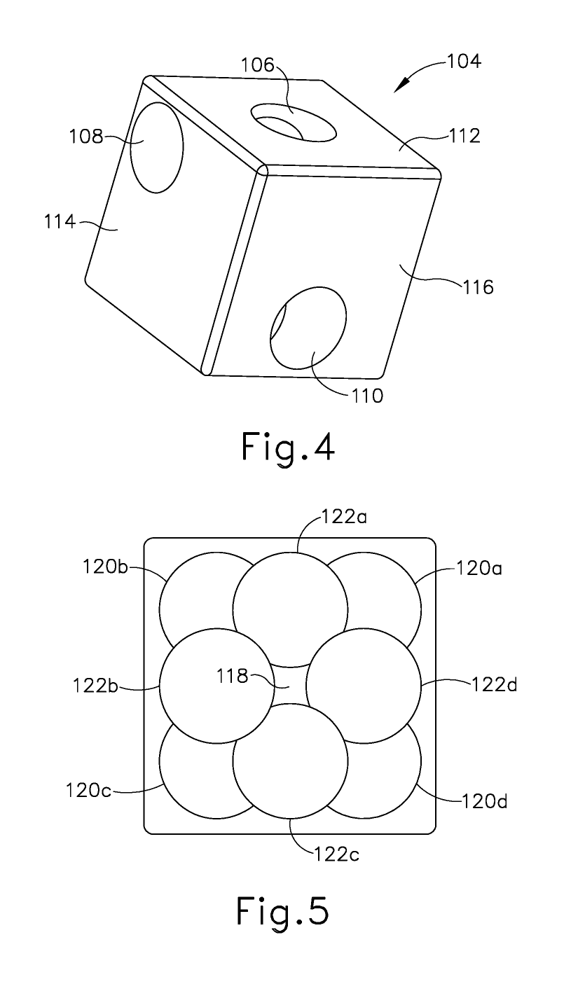

In FIG. 4, guide cube (104) includes a central guide hole (106), a corner guide hole (108), and an off-center guide hole (110) that pass orthogonally to one another between respective opposite pairs of faces (112, 114, 116). By selectively rotating guide cube (104) in two axis, one of pairs of faces (112, 114, 116) may be proximally aligned to an unturned position and then selected proximal face (112, 114, 116) optionally rotated a quarter turn, half turn, or three quarter turn. Thereby, one of nine guide positions (118) (i.e., using central guide hole (106)), (120a-120d) (i.e., corner guide hole (108)), (122a-122d) (i.e., using off-center guide hole (110)) may be proximally exposed as depicted in FIG. 5.

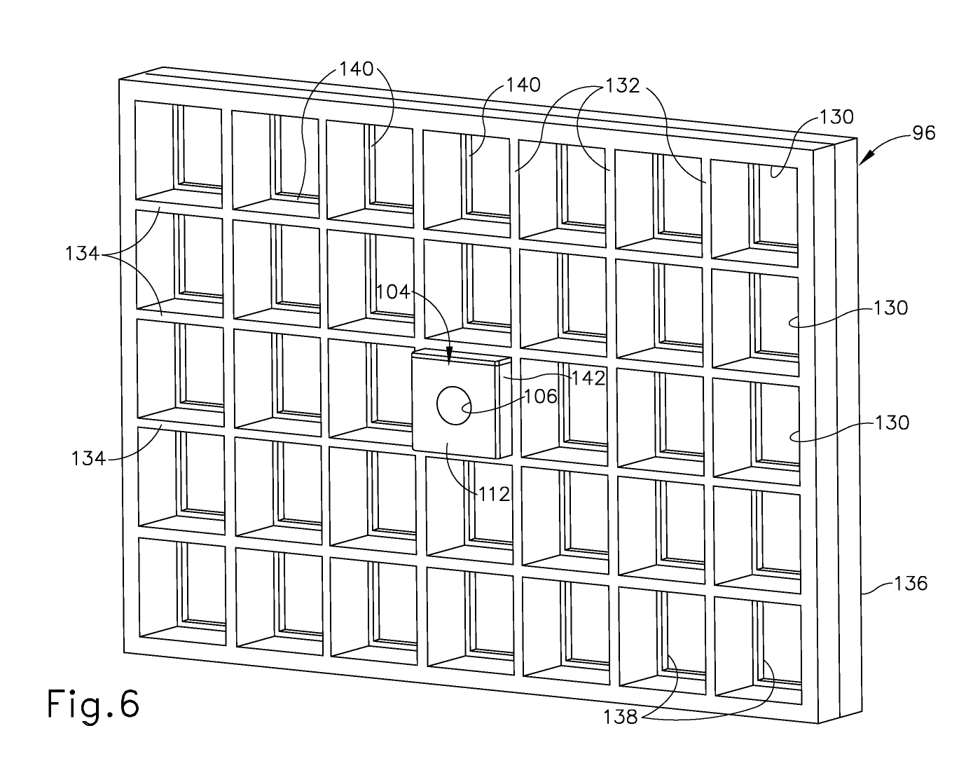

In FIG. 6, two-axis rotatable guide cube (104) is sized for insertion from a proximal side into one of a plurality of square recesses (130) in grid plate (96), which are formed by intersecting vertical bars (132) and horizontal bars (134). Guide cube (104) is prevented from passing through grid plate (96) by backing substrate (136) attached to a front face of grid plate (96). Backing substrate (136) includes respective square opening (138) centered within each square recess (130), forming lip (140) sufficient to capture the front face of guide cube (104), but not so large as to obstruct guide holes (104, 106, 108). The depth of square recesses (130) is less than guide cube (104), thereby exposing a proximal portion (142) of guide cube (104) for seizing and extraction from grid plate (96). It will be appreciated by those of ordinary skill in the art based on the teachings herein that backing substrate (136) of grid plate (96) may be omitted altogether in some versions. In some such versions without backing substrate (136) other features of a guide cube, as will be discussed in more detail below, may be used to securely and removably fit a guide cube within a grid plate. However, such other features may also be used in combination with a grid plate having backing substrate (136), such as grid plate (96), instead of partially or wholly omitting backing substrate (136).

In some other versions, guide cube (104) is replaced with an alternative guide cube or other guide structure that is configured and operable in accordance with at least some of the teachings of U.S. Pat. Pub. No. 2015/0025414, entitled “Biopsy Device Targeting Features,” published Jan. 22, 2015, the disclosure of which is incorporated by reference herein.

V. Exemplary Targeting Set with Dual Latch Locking Mechanism

In some instances it may be desirable for an obturator to be securely attached to a cannula prior to an operator utilizing the targeting set in a medical procedure. Although cannula (94) is configured to slidably receive obturator (92) within lumen (196), obturator (92) may remain insecurely retained in cannula (94) such that securely attaching obturator (92) to cannula (94) may be beneficial to prevent premature detachment of targeting set (89) during active use in a medical procedure. A targeting set that includes corresponding locking features on the obturator and cannula may be beneficial to interlock the components together. Fastening the obturator and cannula together may be desirable to ensure the targeting set is not inadvertently disassembled during the medical procedure.

In assemblies of obturators and cannulas such as targeting set (89) described above, it may be beneficial to provide an interlocking mechanism that is capable of easily locking and unlocking the obturator from the cannula with a single hand. Providing a mechanism that allows an operator to easily engage and disengage the corresponding locking features of the obturator and cannula may be desirable to allow the operator to quickly assembles and disassemble the targeting set without requiring the use of both hands. In other words, it may be desirable to provide a targeting set that can securely fasten the obturator to the cannula, and easily unfasten the obturator from the cannula, through an input feature that can be fully functioned with the use of one hand.

The following description provides various examples of a targeting set including an obturator and cannula that are cooperatively configured to prevent the inadvertent disengagement of the obturator from the cannula during the active use of the targeting set by an operator. Ultimately, the interlocking mechanism of the targeting set may be beneficial to improve patient safety without substantially increasing the amount of effort required by an operator to lock the operator to the cannula. It should be understood that the targeting set described below may be readily incorporated into any of the various targeting sets (89), obturators (92), cannulas (94), guide cubes (104), biopsy devices (14), and biopsy systems (10) described above and in any of the various surgical procedures described in the various references described herein. Other suitable ways in which the below-described obturators and cannulas may be used will be apparent to those of ordinary skill in the art in view of the teachings herein. Except as otherwise described below, targeting set (1089), obturator (1092) and cannula (1094) may be configured and operable just like targeting set (89), obturator (92) and cannula (94), respectively, described above.

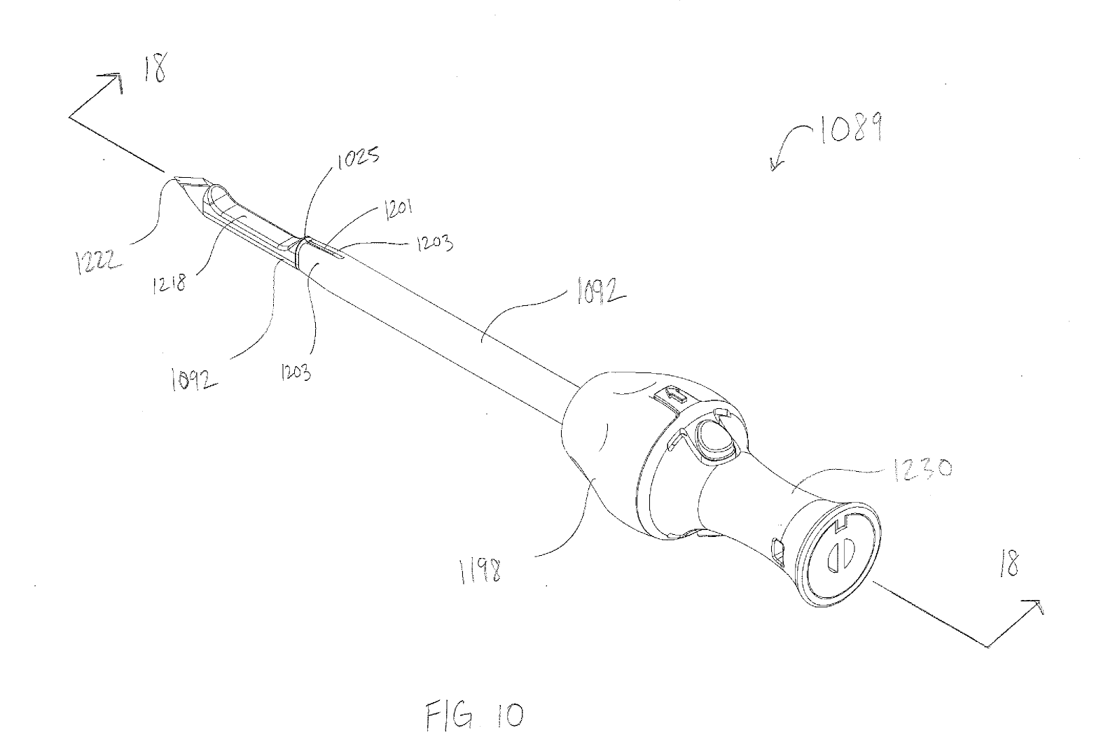

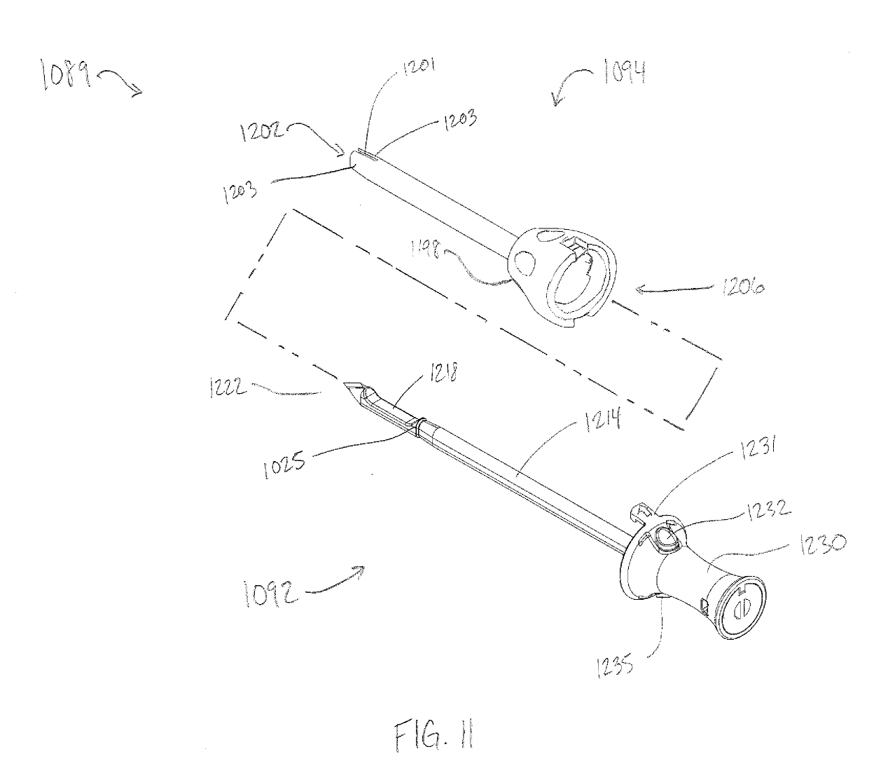

FIG. 10 shows an exemplary alternative targeting set (1089) for use in association with probe (91) as similarly described above with respect to targeting set (89). Targeting set (89) comprises an obturator (1092) and a cannula (1094). Cannula (1094) includes a lumen (1196) extending between a cylindrical hub (1198) and an open distal end (1202). Cannula (1094) generally defines an oval-shaped cross-section that corresponds to an oval-shaped cross-section of obturator (1092). As will be described in greater detail below, this configuration generally provides a rotational locking feature to lock rotation of obturator (1092) relative to cannula (1094) when obturator (1092) is disposed within cannula (1094).

In contrast to cannula (94), which includes lateral aperture (201) at distal end (202), cannula (1094) includes a pair of slots (1201) proximate to open distal end (1202) which defines a pair of sleeves (1203). Slots (1201) are each configured to permit some flexibility of open distal end (1202) to permit cannula (1094) to receive certain features of obturator (1092), as will be described in greater detail below. Sleeves (1203) are configured to deflect inwardly towards lumen (1196) to thereby minimize the interface point of open distal end (1202) along shaft (1214) when obturator (1092) is coupled to cannula (1094). In other words, sleeves (1203) are naturally inclined to extend inwardly towards each other such that the interface point between open distal end (1202) and shaft (1214) when obturator (1092) and cannula (1094) are coupled together is a substantially level plane. Slots (1201) are sized and shaped to form an elliptical or oval opening along cannula (1094) to thereby allow sleeves (1203) to flexibly deflect inwards or outwards relative to lumen (1196) by expanding or contracting slots (1203). Slots (1201) and sleeves (1203) are configured and operable in accordance with the teachings of U.S. App. No. 62/509,496, entitled “MRI Targeting Set With Improved Targeting Sleeve,” filed on May 22, 2017, the disclosure of which is incorporated by reference herein.

Obturator (1092) includes a shaft (1214) extending between an obturator seal cap (1230) or endpiece, positioned on a proximal end of shaft (1214), and a piercing tip (1222), positioned on a distal end of shaft (1214). Shaft (1214) is longitudinally sized such that a piercing tip (1222) extends out of open distal end (1202) of cannula (1094). The lateral cross-section of obturator (1092) defines a generally oval-shaped cross-section. This oval-shaped cross-section generally corresponds to an oval-shape defined by cannula (1194). As described above, this corresponding oval-shape of both obturator (1092) and cannula (1094) generally provide a rotational lock feature such that obturator (1092) is rotationally secured relative to cannula (1094) when obturator (1092) is inserted into cannula (1094). Although both cannula (1094) and obturator (1092) are described herein as including an oval-shaped cross-section, it should be understood that in other examples cannula (1094) and obturator (1092) may have numerous alternative shapes. For instance, in some examples cannula (1094) and obturator (1092) have a cylindrical shape, an egg-shape, or any other suitable shape as will be apparent to those of ordinary skill in the art in view of the teachings herein.

Obturator (1092) further includes an imageable side notch (1218) proximal to piercing tip (1222) that is configured to extend beyond open distal end (1202) of cannula (1094) when obturator (1092) is slidably received within cannula (1094). Obturator (1092) further includes a neck (1025) on a distal end of shaft (1214). Neck (1025) is positioned along shaft (1214) proximally relative to piercing tip (1222). Neck (1025) defines extends inwardly relative to an exterior surface of shaft (1214) along the outer perimeter of shaft (1214). Thus, neck (1025) defines an indented surface relative to the surface of shaft (1214). Neck (1025) is configured to receive open distal end (1202) of cannula (1094) with obturator (1092) slidably inserted into cannula (1094) such that open distal end (1202) is positioned along shaft (1214) proximally relative to neck (1025).

In the present example, the inward extension of neck (1025) from the surface of shaft (1214) is configured to be at least equal to or greater than a thickness of cannula (1094). Neck (1025) is configured to cover or shield open distal end (1202) of cannula (1194) when obturator (1092) is slidably inserted through cannula (1094) to thereby minimize the interface between obturator (1092) and cannula (1094). In at least some examples, neck (1025) is configured and operable in accordance with the teachings of U.S. App. No. 62/509,496, entitled “MRI Targeting Set With Improved Targeting Sleeve,” filed on May 22, 2017, the disclosure of which is incorporated by reference herein.

In contrast to obturator (92), which includes fluid lumen (216) defined by shaft (214), shaft (1214) of obturator (1092) is formed of a solid material and does not include a proximal port in fluid communication with a fluid lumen. Furthermore, obturator (92) includes locking feature (226) on obturator thumbwheel cap (224) and a separate locking feature (232) on obturator seal cap (230), whereas obturator (1092) includes a single obturator seal cap (1230) including a dual latch locking mechanism (1233, 1236) as will be described in greater detail below.

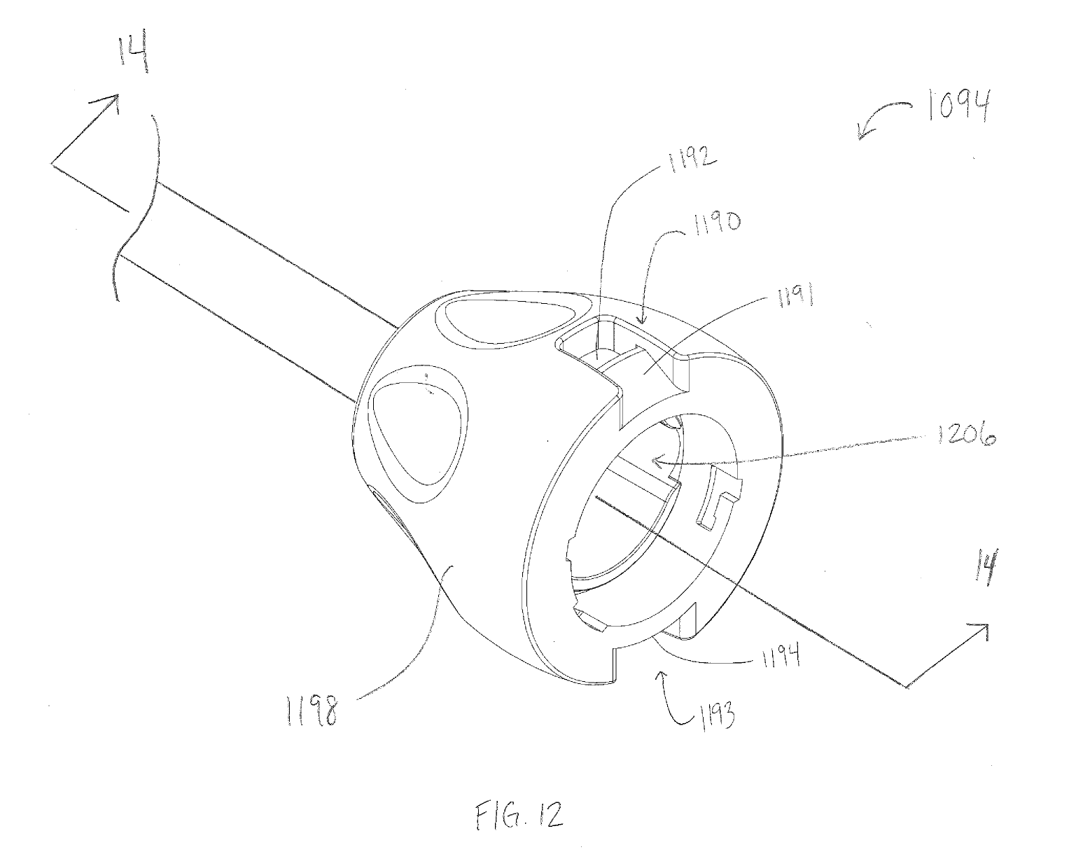

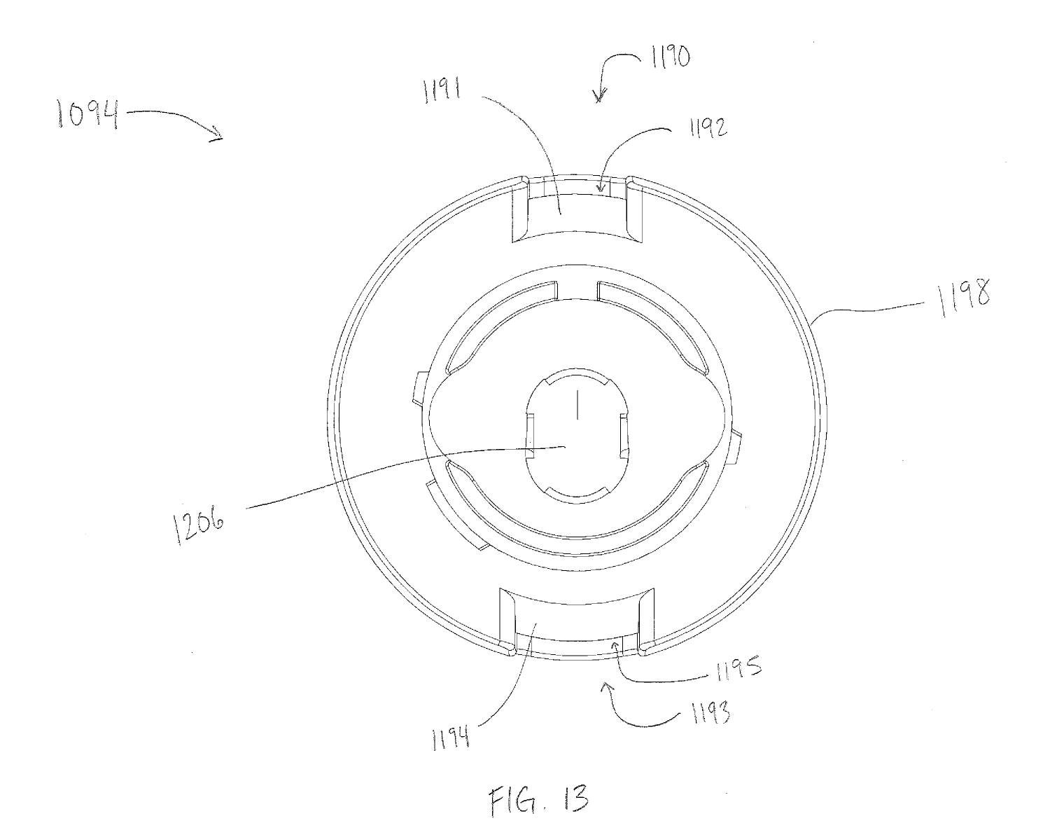

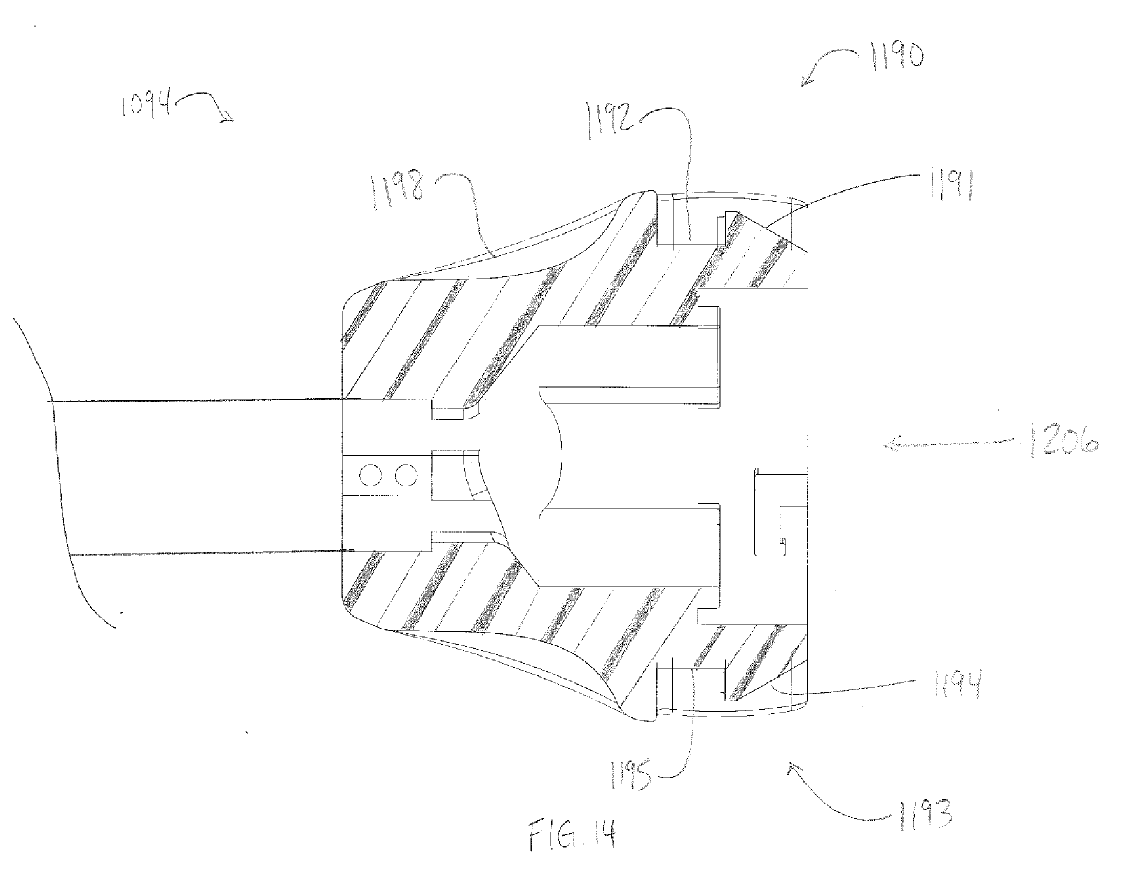

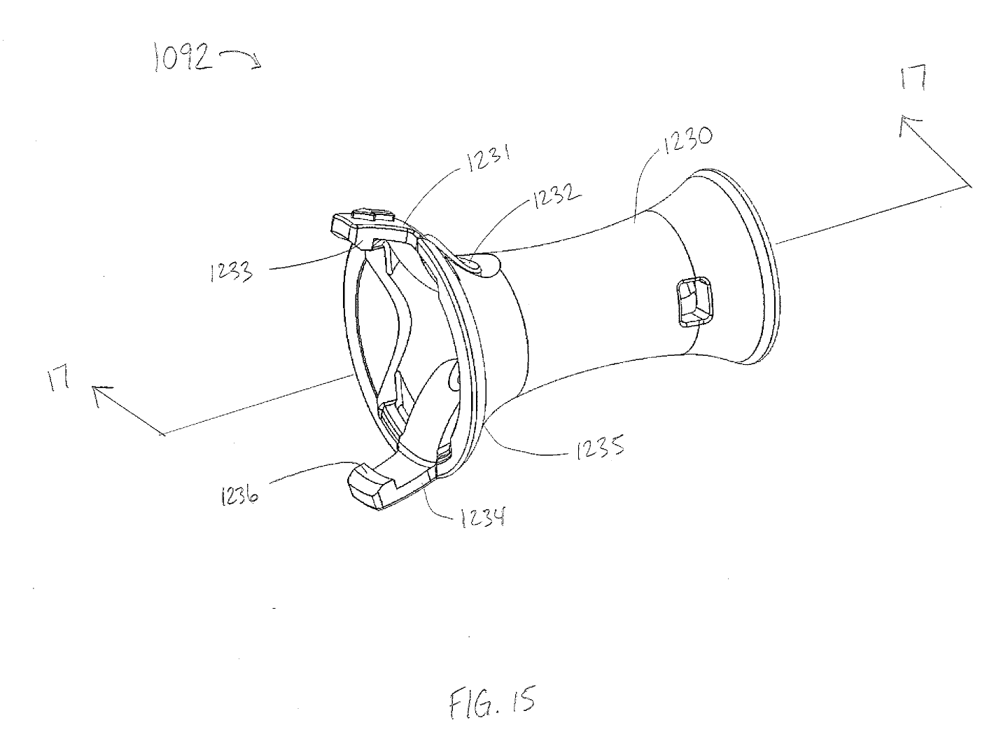

As best seen in FIG. 11, cylindrical hub (1198) of cannula (1094) includes an interior recess (1206) that is in communication with lumen (1196) and is configured to receive obturator (1092). As best seen in FIG. 12, cylindrical hub (1198) further includes a first receiving notch (1191) and a second receiving notch (1193) positioned along the outer perimeter of cylindrical hub (1198). As further seen in FIG. 12, catches or receiving notches (1190, 1193) include a chamfered ramp (1191, 1194) and a retaining pocket (1192, 1195), respectively. An upper catch or notch (1190) is configured to have a narrower chamfered ramp (1191) and retaining pocket (1192) than chamfered ramp (1194) and retaining pocket (1195) of an opposite lower catch or notch (1193). In other words, lower chamfered ramp (1194) is wider than upper chamfered ramp (1191) and lower retaining pocket (1195) is wider than upper retaining pocket (1192). As will be described in greater detail below, this difference in configuration of chamfered ramps (1191, 1194) and retaining pockets (1192, 1195) generally promotes a keyed relationship between obturator (1092) and cannula (1094) to provide locking in a single orientation. Notwithstanding lower notch (1193) being wider than upper notch (1190), notches (1190, 1193) are axially aligned about interior recess (1206) of cylindrical hub (1198), as seen in FIG. 13. As best shown in FIG. 14, chamfered ramps (1191, 1194) are configured to incline towards retaining pockets (1194, 1195) such that retaining pockets (1194, 1195) are recessed into cylindrical hub (1198), towards interior recess (1206), relatively lower than chamfered ramps (1191, 1194).

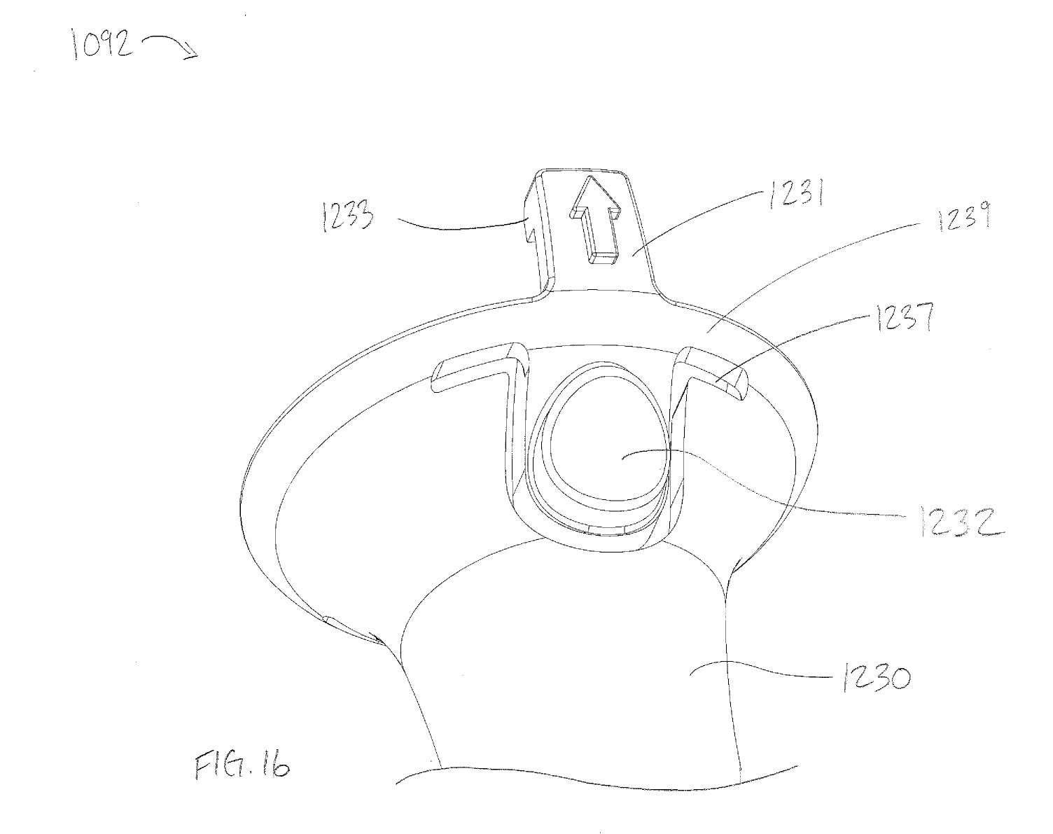

FIG. 15 shows obturator seal cap (1230) in greater detail. As can be seen, obturator seal cap (1230) includes dual latching mechanism in the form of an upper arm (1231) and a lower arm (1234). The dual latching mechanism of obturator seal cap (1230) is generally configured to provide selective coupling of obturator (1092) to cannula (1094). This selective coupling operation is generally provided by a rocker arm configuration that is defined by the combination of seal cap (1230) and arms (1231, 1234). Upper arm (1231) and lower arm (1234) are positioned along the outer perimeter of obturator seal cap (1230). As will be described in greater detail below, upper arm (1231) is positioned along on obturator seal cap (1230) to correspond with the position of upper notch (1190) on cylindrical hub (1198) of cannula (1094). Similarly, lower arm (1234) is positioned along on obturator seal cap (1230) to correspond with the position of lower notch (1193) on cylindrical hub (1198) of cannula (1094). As will be described in greater detail below, upper arm (1231) is sized and shaped to only be received by upper notch (1190) and lower arm (1234) is sized and shaped to only be received within lower notch (1193).

As further seen in FIG. 15, upper arm (1231) includes an upper latch (1233) extending laterally from upper arm (1231) towards lower arm (1234). Correspondingly, lower arm (1234) includes a lower latch (1236) extending laterally from lower arm (1234) towards upper arm (1231). Latches (1233, 1236) are integrally formed with arms (1231, 1234), respectively. As will be described in greater detail below, upper latch (1233) is sized and shaped to be received within upper retaining pocket (1192), while lower latch (1236) is sized and shaped to correspond with lower retaining pocket (1195) only. In other words, upper latch (1233) is configured to not able to be received within lower retaining pocket (1195). Similarly, lower latch (1236) is configured to not be able to be received within upper retaining pocket (1192).

Obturator seal cap (1230) further includes an upper actuator or input feature (1232) and a lower actuator or input feature (1235) such that upper input feature (1232) is integrally formed with the upper arm (1231) and the lower input feature (1235) is integrally formed with the lower arm (1234). Upper input feature (1232) is configured to selectively pivot upper arm (1231) from an engaged position to a disengaged position when an operator operatively manipulates upper input feature (1232). Correspondingly, lower input feature (1235) is configured to selectively pivot lower arm (1234) from an engaged position to a disengaged positioned through the manipulation of lower input feature (1235).

Accordingly, upper input feature (1232) is configured to selectively pivot upper arm (1231) and upper latch (1233) relative to obturator seal cap (1230) upon manipulation of upper input feature (1232) by an operator. Similarly, lower input feature (1235) is configured to selectively pivot lower arm (1234) and lower latch (1236) relative to obturator seal cap (1230) upon manipulation of lower input feature (1234). Input features (1232, 1235) are generally configured to convert an inward lateral force into an outward lateral force such that the inward depression of input features (1232, 1235) into obturator seal cap (1230) provides for the outward extension of latches (1233, 1236) through the pivoting of arms (1231, 1234). As will be described in greater detail below, to maintain locking once an operator has manipulated input features (1232, 1235), arms (1231, 1234) are generally configured to be resiliently biased towards a locked position. Thus, once an operator releases input features (1232, 1235), arms (1231, 1234) will automatically return to their pre-manipulated position.

As best seen in FIG. 15, upper latch (1233) has a narrower width than lower latch (1236) such that upper latch (1233) is configured to be inserted into upper notch (1190) and lower latch (1236) is configured to be inserted into lower notch (1193). In other words, the narrow width of upper notch (1190) is configured to correspond with the narrow width of upper latch (1233) such that lower notch (1193) is not able to be received within upper notch (1190). Although upper latch (1233) may have a size sufficient to be received within lower latch (1236), it should be understood that the dissimilar fit between lower latch and upper notch (1190) will otherwise prevent coupling between upper latch (1233) and lower notch (1193), and lower latch (1236) and upper notch (1190).

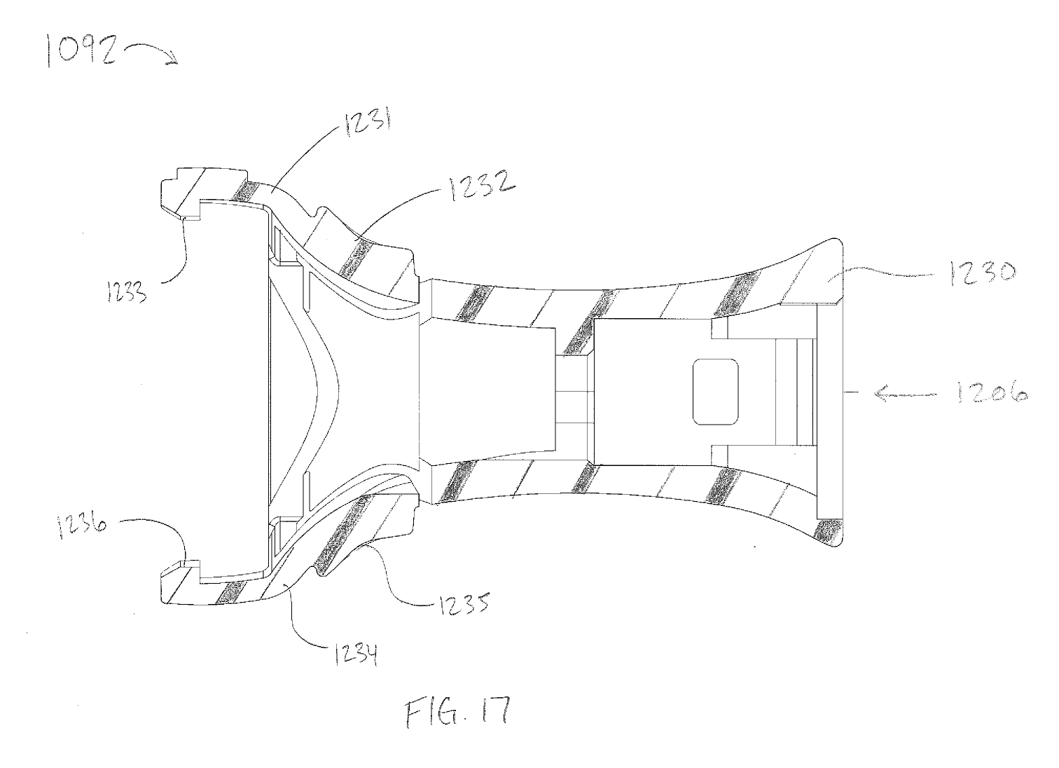

Obturator seal cap (1230) further includes an upper aperture (1237) and a linkage portion (1239). Linkage portion (1239) extends around the outer perimeter of obturator seal cap (1230) and is positioned between arms (1231, 1234) and the remaining portion of obturator seal cap (1230). As seen in FIG. 16, upper aperture (1237) is positioned around upper input feature (1232) except for at linkage portion (1239). Upper aperture (1237) is configured to provide for the flexible depression of upper input feature (1232) into obturator seal cap (1230). In this instance, upper aperture (1237) is not positioned at linkage portion (1239) to maintain the integral connection of upper input feature (1232) with upper arm (1231). Upper aperture (1237) is sized and shaped to allow for the operative manipulation of upper input feature (1232) into obturator seal cap (1230) to thereby pivot upper arm (1231) from the engaged position to the disengaged position. Similarly, obturator seal cap (1230) includes a lower aperture (1238) positioned around lower input feature (1235) except for at linkage portion (1239). Lower aperture (1238) is configured to provide for the flexible depression of lower input feature (1235) into obturator seal cap (1230). Similar to upper aperture (1237), lower aperture (1238) is not positioned at linkage portion (1239) to maintain the integral connection of lower input feature (1235) with lower arm (1234). Lower aperture (1238) is sized and shaped to allow for the selective manipulation of lower input feature (1235) relative to obturator seal cap (1230) to thereby pivot lower arm (1234) between the engaged and disengaged position.

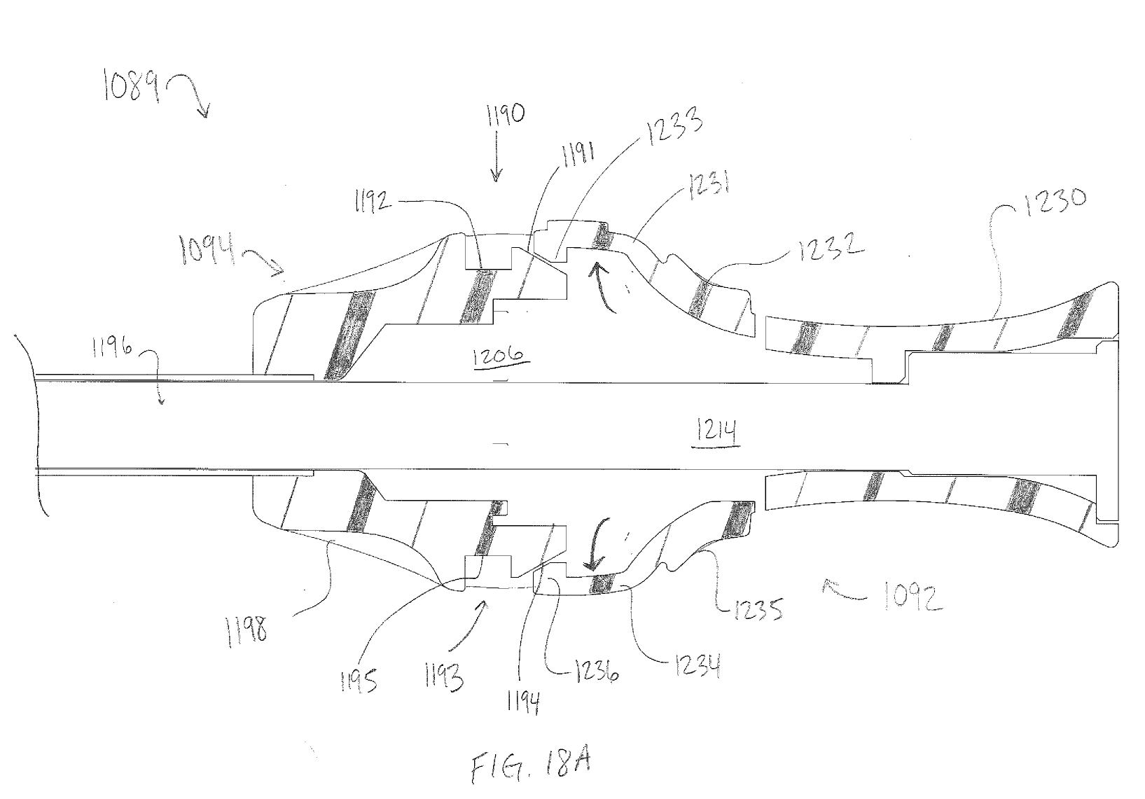

As seen in FIG. 17, upper arm (1231), upper input feature (1232) and upper latch (1233) are integrally formed with each other and obturator seal cap (1230) such that the selective manipulation of any causes a simultaneous movement of the other components. Similarly, lower arm (1234), lower input feature (1235) and lower latch (1236) are integrally formed, thus lower latch (1236) and lower arm (1234) are configured to pivot relative to obturator seal cap (1230) upon the lateral depression of lower input feature (1235). It should be understood that notches (1190, 1193) may be positioned along cylindrical hub (1198), while and latches (1233, 1236), arms (1231, 1234) and input features (1232, 1235) may be positioned correspondingly along obturator seal cap (1230).

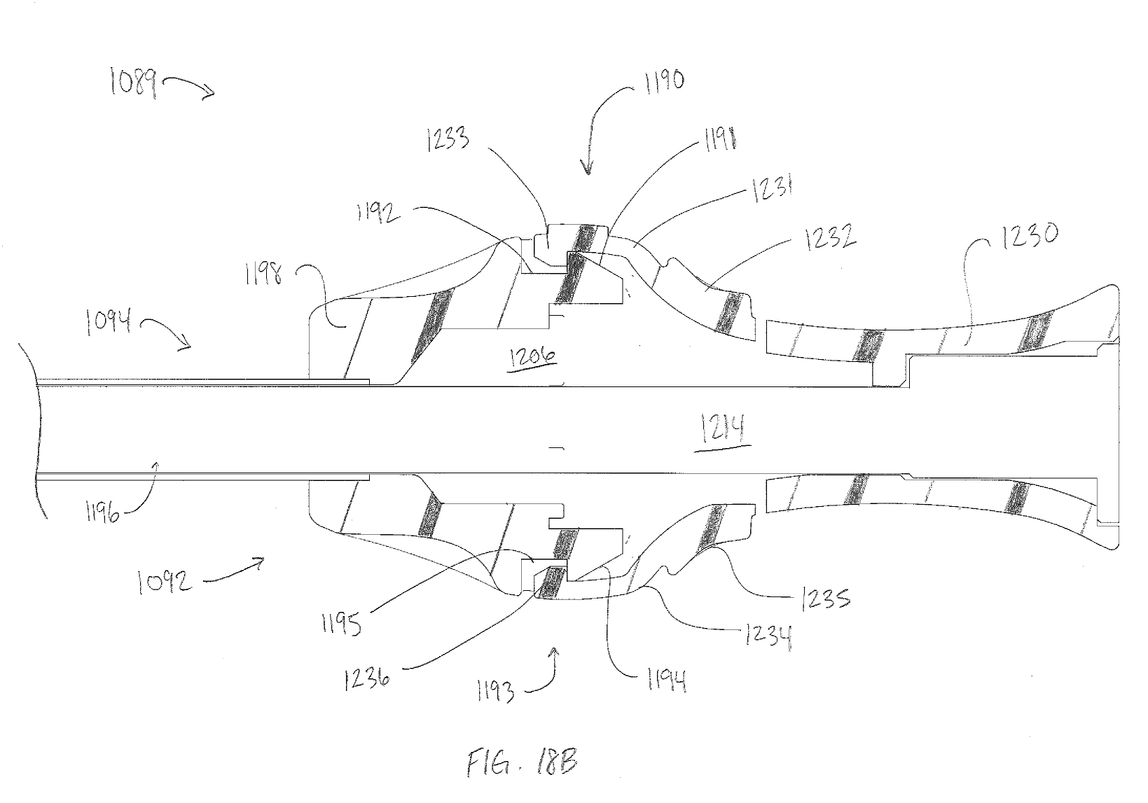

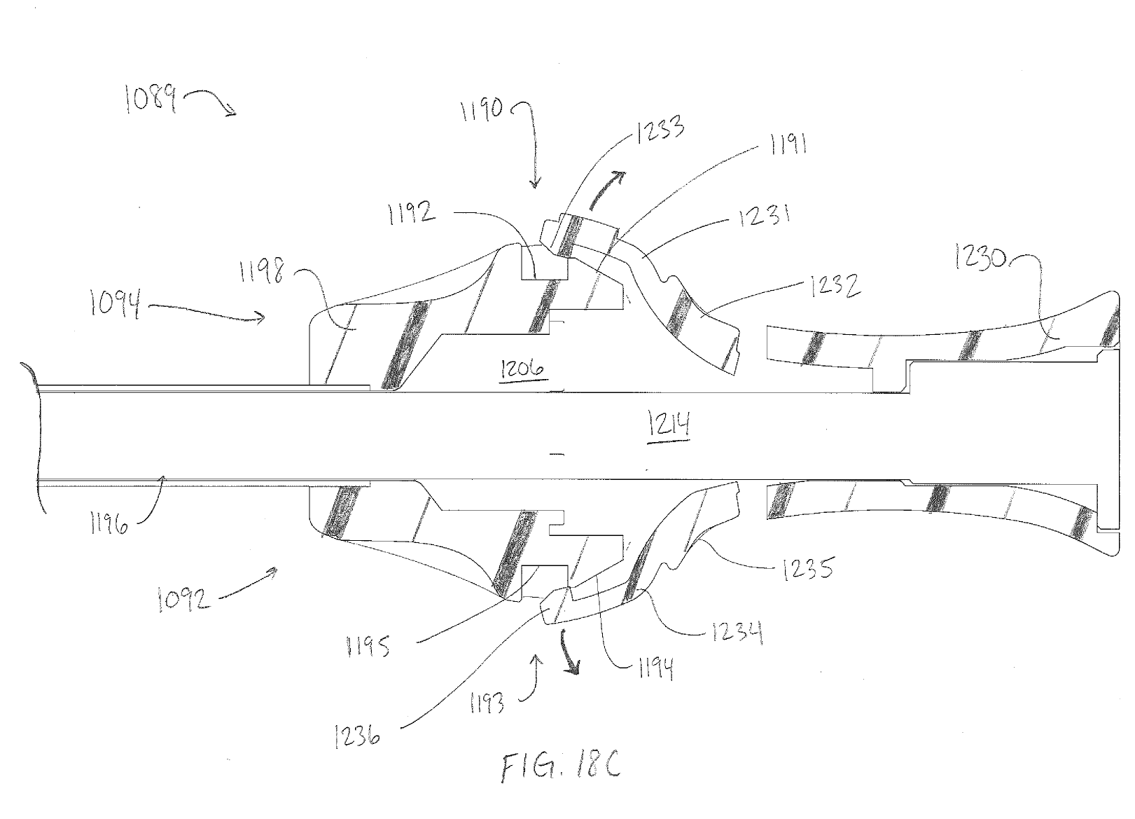

In the present example, as shown in FIGS. 18A-18C, obturator (1092) securely couples to cannula (1094) through the interlocking engagement of obturator seal cap (1230) and cylindrical hub (1198). Particularly, obturator (1092) becomes fixedly attached to cannula (1094) through the coupling of latches (1233, 1236) with notches (1190, 1193), respectively. As seen in FIG. 18A, shaft (1214) of obturator (1092) is slidably inserted through interior recess (1206) of cannula (1094) and into lumen (1196) until latches (1233, 1236) encounter notches (1190, 1193) at chamfered ramps (1191, 1194). In this instance, upper latch (1233) is aligned with upper notch (1190) and lower latch (1236) is aligned with lower notch (1193). Although not shown, in the event lower latch (1236) is aligned with upper notch (1190) and upper latch (1233) is aligned with lower notch (1193), obturator (1092) will be prevent from coupling with cannula (1094) as lower latch (1236) would not fit into upper chamfered ramp (1191) or upper retaining pocket (1192). Thus, the corresponding sizes of notches (1190, 1193) and latches (1233, 1236) serve to indicate to an operator whether obturator seal cap (1230) has the correct rotational alignment with cylindrical hub (1198) to effectively couple obturator (1092) to cannula (1094).

With latches (1233, 1236) encountering chamfered edges (1191, 1194), respectively, the continued distal advancement of shaft (1214) into lumen (1196) will cause arms (1231, 1234) to pivot outward relative to shaft (1214) as latches (1233, 1236) engage the inclined surface of chamfered ramps (1191, 1194). Latches (1233, 1236) continue to advance along chamfered ramps (1191, 1194) as shaft (1214) and obturator (1092) are slidably inserted into cannula (1094) until latches (1233, 1236) are received within retaining pockets (1192, 1195), respectively, as seen in FIG. 18B. In this instance, arms (1231, 1234) pivot inwardly towards shaft (1214) as latches (1233, 1236) are received within and become securely fixed in retaining pockets (1192, 1195). With latches (1233, 1236) engaged against retaining pockets (1192, 1195), obturator (1092) is securely coupled to cannula (1094) and is prevented from being axially withdrawn from cannula (1094).

To selectively decouple obturator (1092) from cannula (1094), an operator may selectively manipulate input features (1232, 1235) to actuate arms (1231, 1234) and thereby release latches (1233, 1236) from retaining pockets (1192, 1195). In particular, as seen in FIG. 18C, exerting an inward force onto input features (1232, 1235), relative to shaft (1214), causes input features (1232, 1235) to depress into obturator seal cap (1230) due to input features (1232, 1235) being surrounded by apertures (1237, 1238), respectively. Due to input features (1232, 1235) being integrally formed with arms (1231, 1234), respectively, the inward depression of input features (1232, 1235) provides for the upward pivot of arms (1231, 1234) and latches (1233, 1236) attached therein. With latches (1233, 1236) pivoted about arms (1231, 1234), latches (1233, 1236) are effectively removed out of retaining pockets (1192, 1195). In this instance, shaft (1214) may be proximally withdrawn from lumen (1196) to effectively disengage latches (1233, 1236) from notches (1190, 1195) and thereby decouple obturator (1092) from cannula (1094).

VI. Exemplary Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### EXAMPLE 1

A targeting set for use with positioning a biopsy device within a patient, the targeting set comprising: (a) an obturator including a first shaft, a tissue receiving feature, and a seal cap, (b) a cannula including a second shaft, an open distal end, and a proximal hub; wherein the second shaft defines an internal lumen configured to receive the first shaft; wherein the seal cap includes at least one latching mechanism; wherein the proximal hub includes at least one receiving mechanism such that the at least one latching mechanism is configured to releasably engage the at least one receiving mechanism; wherein the tissue receiving feature is configured to extend beyond the open distal end with the releasable engagement of the at least one latching mechanism and the at least one receiving mechanism; and wherein the obturator is configured to securely attach to the cannula with the releasable engagement of the at least one latching mechanism and the at least one receiving mechanism.

### EXAMPLE 2

The targeting set of Example 1, wherein the latching mechanism includes a latch, an arm, and an input feature.

### EXAMPLE 3

The targeting set of Example 2, wherein the latch, arm, and input feature are integrally formed.

### EXAMPLE 4

The targeting set of Example 2 through Example 3, wherein the input feature is configured to pivot the arm and latch such that pivoting the arm and the latch disengages the latching mechanism from the receiving mechanism.

### EXAMPLE 5

The targeting set of Example 2 through Example 4, wherein the latching mechanism includes at least two latches, at least two arms, and at least two input features.

### EXAMPLE 6

The targeting set of Example 5, wherein the at least two input features are buttons.

### EXAMPLE 7

The targeting set of Example 1 through Example 5, wherein the receiving mechanism includes a chamfered ramp and a retaining pocket.

### EXAMPLE 8

The targeting set of Example 6, wherein the chamfered ramp is configured to deflect the latch laterally and pivot the arm outwardly.

### EXAMPLE 9

The targeting set of Example 6 through Example 7, wherein the retaining pocket is configured to receive the latch such that the receiving mechanism securely engages the locking mechanism to thereby releasably attach the obturator to the cannula.

### EXAMPLE 10

The targeting set of Example 9, wherein the receiving mechanism includes at least two chamfered ramps and at least two retaining pockets.

### EXAMPLE 11

The targeting set of Example 5, wherein the at least two latches have different widths.

### EXAMPLE 12

The targeting set of Example 10, wherein the at least two chamfered ramps have different widths.

### EXAMPLE 13

The targeting set of Example 10 through Example 11, wherein the at least two retaining pockets have different widths.

### EXAMPLE 14

The targeting set of Example 11 through Example 13, wherein a first width of one of the at least two latches corresponds with a second width of one of the at least two chamfered ramps.

### EXAMPLE 15

The targeting set of Example 14, wherein a third width of the other at least two latches corresponds with a fourth width of the other at least two chamfered ramps.

### EXAMPLE 16

The targeting set of Example 1, further including a vacuum assembly, wherein the vacuum assembly comprises: (i) a pressure source, and (ii) a valve assembly, wherein the valve assembly is responsive to the pressure source to induce vacuum in the tissue receiving feature of the obturator.

### EXAMPLE 17

The targeting set of Example 1, wherein the cannula further comprises a lateral aperture, wherein the lateral aperture of the cannula is configured to align with the tissue receiving feature of the obturator when the first shaft is received within the internal lumen of the second shaft.

### EXAMPLE 18

A biopsy system, comprising: (a) a biopsy device, wherein the biopsy device comprises: (i) a body, (ii) a needle, and (iii) a cutter, wherein the needle extends from the body to collect tissue samples using the cutter; and (b) a targeting set, wherein the targeting set comprises: (i) an obturator having a first shaft, a sharp distal tip, a distal neck, and a proximal cap, and (ii) a cannula having a second shaft, an open distal end, a pair of distal sleeves, and a proximal hub; wherein the cannula defines a lumen, wherein the lumen is configured to separately receive the needle of the biopsy device and the obturator; wherein the proximal cap includes one or more latching mechanisms; wherein the proximal hub includes one or more receiving mechanisms configured to releasably engage the one or more latching mechanisms; wherein the tissue receiving feature is configured to extend beyond the open distal end when the one or more latching mechanism engages the one or more receiving mechanisms such that the obturator is configured to securely attach to the cannula; and wherein the pair of distal sleeves are configured to be received by the distal neck when the one or more latching mechanisms engages the one or more receiving mechanisms such that an interface portion between the neck and the open distal end is a substantially level plane.

V. Conclusion

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Embodiments of the present invention have application in conventional endoscopic and open surgical instrumentation as well as application in robotic-assisted surgery.

By way of example only, embodiments described herein may be processed before surgery. First, a new or used instrument may be obtained and if necessary cleaned. The instrument may then be sterilized. In one sterilization technique, the instrument is placed in a closed and sealed container, such as a plastic or TYVEK bag. The container and instrument may then be placed in a field of radiation that can penetrate the container, such as gamma radiation, x-rays, or high-energy electrons. The radiation may kill bacteria on the instrument and in the container. The sterilized instrument may then be stored in the sterile container. The sealed container may keep the instrument sterile until it is opened in a medical facility. A device may also be sterilized using any other technique known in the art, including but not limited to beta or gamma radiation, ethylene oxide, or steam.

Embodiments of the devices disclosed herein can be reconditioned for reuse after at least one use. Reconditioning may include any combination of the steps of disassembly of the device, followed by cleaning or replacement of particular pieces, and subsequent reassembly. In particular, embodiments of the devices disclosed herein may be disassembled, and any number of the particular pieces or parts of the devices may be selectively replaced or removed in any combination. Upon cleaning and/or replacement of particular parts, embodiments of the devices may be reassembled for subsequent use either at a reconditioning facility, or by a surgical team immediately prior to a surgical procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/we claim:

1. A targeting set for use with positioning a biopsy device relative to a patient, the targeting set comprising:
(a) an obturator having a seal cap;
(b) a sleeve having a hub, wherein the sleeve defines a lumen configured to receive the obturator through the hub; and
(c) a latching assembly, wherein the latching assembly includes an actuator arm attached to the seal cap and a catch positioned in the sleeve hub, the actuator arm having a proximal end and a distal end adapted to mate with the catch, wherein the proximal end of the actuator arm is configured to move radially inwardly to produce a radially outward movement of the distal end of the actuator arm to unlock the sleeve from the obturator.

2. The targeting set of claim 1, wherein the proximal end of the actuator arm is biased to move radially inwardly to keep the distal end of the actuator arm engaged with the catch.

3. The targeting set of claim 1, wherein the catch has a ramp that extends radially outwardly in the distal direction, wherein the ramp is configured to engage the distal end of the actuator arm to displace the distal end of the actuator arm radially outwardly.

4. The targeting set of claim 1, wherein the proximal end of the actuator arm is biased to move radially inwardly to keep the distal end of the actuator arm engaged with the catch, wherein the catch includes a ramp that extends radially outwardly in the distal direction, wherein the ramp is configured to engage the distal end of the actuator arm to displace the distal end of the actuator arm radially outwardly against the bias of the proximal end of the actuator arm.

5. The targeting set of claim 1, wherein the latching assembly is a first latching assembly, wherein the targeting set further comprises a second latching assembly including an actuator arm attached to the seal cap and a catch positioned in the sleeve hub.

6. The targeting set of claim 1, wherein the latching assembly is a first latching assembly, wherein the targeting set further comprises a second latching assembly including an actuator arm attached to the seal cap and a catch positioned in the sleeve hub, wherein the proximal end of the actuator arm of both the first latching assembly and the second latching assembly is biased to move radially inwardly to keep the distal end of the actuator arm of each respective latching assembly engaged with each respective catch.

7. The targeting set of claim 1, wherein the latching assembly is a first latching assembly, wherein the targeting set further comprises a second latching assembly including an actuator arm attached to the seal cap and a catch positioned in the sleeve hub, wherein the catch of each of the first latching assembly and the second latching assembly has a ramp that extends radially outwardly in the distal direction, wherein the ramp of each catch is configured to engage the distal end of a respective actuator arm to displace the distal end of the respective actuator arm radially outwardly.

8. The targeting set of claim 1, wherein the latching assembly is a first latching assembly, wherein the targeting set further comprises a second latching assembly including an actuator arm attached to the seal cap and a catch positioned in the sleeve hub, wherein the proximal end of the actuator arm of both the first latching assembly and the second latching assembly is biased to move radially inwardly to keep the distal end of the actuator arm of each respective latching assembly engaged with each respective catch, wherein the catch of each of the first latching assembly and the second latching assembly has a ramp that extends radially outwardly in the distal direction, wherein the ramp of each catch is configured to engage the distal end of a respective actuator arm to displace the distal end of the respective actuator arm radially outwardly against the bias of the proximal end.

9. The targeting set of claim 1, the latching assembly is a first latching assembly, wherein the targeting set further comprises a second latching assembly including an actuator arm attached to the seal cap and a catch positioned in the sleeve hub, wherein the catch of the second latching assembly is configured to only receive the actuator arm of the second latching assembly and not the actuator arm of the second latching assembly.

10. The targeting set of claim 1, wherein the latching assembly is a first latching assembly, wherein the targeting set further comprises a second latching assembly including an actuator arm attached to the seal cap and a catch positioned in the sleeve hub, wherein the catch of the second latching assembly defines a width, wherein the width of the catch of the second latching assembly is greater than a width defined by the catch of the first latching assembly.

11. The targeting set of claim 1, wherein the latching assembly is a first latching assembly, wherein the targeting set further comprises a second latching assembly including an actuator arm attached to the seal cap and a catch positioned in the sleeve hub, wherein the actuator arm of the first latching assembly is positioned on an opposite side of the seal cap relative to the actuator arm of the second latching assembly,
wherein the proximal end of the actuator arm of both the first latching assembly and the second latching assembly is biased to move radially inwardly to keep the distal end of the actuator arm of each respective latching assembly engaged with each respective catch,
wherein the catch of each of the first latching assembly and the second latching assembly has a ramp that extends radially outwardly in the distal direction,
wherein the ramp of each catch is configured to engage the distal end of a respective actuator arm to displace the distal end of the respective actuator arm radially outwardly against the bias of the proximal end, and
wherein the ramp of the catch of the first latching assembly is wider than the ramp of the catch of the second latching assembly.

12. The targeting set of claim 1, wherein a portion of the actuator arm protrudes distally from the seal cap of the obturator.

13. The targeting set of claim 1, wherein the catch is recessed into a portion of the hub of the sleeve.

14. A targeting set for use with positioning a biopsy device relative to a patient, the targeting set comprising:
(a) an obturator;
(b) a sleeve, wherein the sleeve defines a lumen configured to receive the obturator; and
(c) a latching assembly, wherein the latching assembly is configured to fasten the obturator to the sleeve, wherein the latching assembly includes a first fastener and a second fastener, wherein the first fastener and the second fastener each include a latch and an actuator, respectively, wherein each actuator is configured to move inwardly to produce outwardly oriented movement of each latch, wherein the latching assembly is configured to engage a portion of the sleeve.

15. The targeting set of claim 14, wherein the obturator includes an elongate shaft and a hub oriented at a proximal end of the shaft, wherein the first fastener and the second fastener of the latching assembly are each secured to the hub, wherein the sleeve includes an elongate cannula and a hub disposed at the proximal end of the cannula, wherein the hub of the obturator is configured to fasten to the hub of the cannula.

16. The targeting set of claim 14, wherein the obturator includes an elongate shaft and a hub oriented at a proximal end of the shaft, wherein the first fastener and the second fastener of the latching assembly are each secured to the hub, wherein the sleeve includes an elongate cannula and a hub disposed at the proximal end of the cannula, wherein the hub of the cannula includes a first recess and a second recess, wherein the first recess is configured to receive the first fastener, wherein the second recess is configured to receive the second fastener.

17. The targeting set of claim 14, wherein the obturator includes an elongate shaft and a hub oriented at a proximal end of the shaft, wherein the first fastener and the second fastener of the latching assembly are each secured to the hub, wherein the sleeve includes an elongate cannula and a hub disposed at the proximal end of the cannula, wherein the hub of the cannula includes a first recess and a second recess, wherein the first recess is configured to receive the first fastener, wherein the second recess is configured to receive the second fastener, wherein the first fastener and the second fastener are each resiliently biased towards the first recess and the second recess, respectively.

18. A targeting set, the targeting set comprising:
(a) an obturator;
(b) a sleeve defining a lumen configured to receive at least a portion of the obturator; and
(c) a dual latching mechanism including a pair of rocker arms and a pair of catches, wherein the dual latching mechanism is configured to selectively couple the obturator to the sleeve, wherein each rocker arm of the pair of rocker arms includes a lock, wherein each lock is configured for receipt in a corresponding catch of the pair of catches, wherein each rocker arm is configured to receive an inwardly oriented input to produce outwardly oriented movement of each respective lock.

19. The targeting set of claim 18, wherein the dual latching mechanism further includes a ramp associated with each catch, wherein each ramp is configured to pivot a corresponding rocker arm when the obturator is inserted into the sleeve.

20. The targeting set of claim 18, wherein each rocker arm of the pair of rocker arms is resiliently biased towards a locking position, wherein the dual latching mechanism further includes a ramp associated with each catch, wherein each ramp is configured to pivot a corresponding rocker arm away from the locking position when the obturator is inserted into the sleeve.
